(19) United States
(12) Patent Application Publication
(10) Pub. No.: US 2023/0120830 A1
(43) Pub. Date: Apr. 20, 2023

(54) Spinal Implant with a Magnesium-Phosphate Three-Dimensional Porosity Structure

(71) Applicant: Bone Solutions, Inc., Colleyville, TX (US)

(72) Inventor: Frankie L. Morris, Colleyville, TX (US)

(21) Appl. No.: 17/969,257

(22) Filed: Oct. 19, 2022

Related U.S. Application Data
(60) Provisional application No. 63/257,640, filed on Oct. 20, 2021.

(51) Int. Cl. A61F 2/44

(52) U.S. Cl. CPC A61F 2/4455; A61F 2002/30062

(57) ABSTRACT

The present disclosure relates to a spinal implant for insertion between two adjacent vertebrae. The spinal implant includes a frame sized to be inserted between the two adjacent vertebrae. The spinal implant also includes a lattice structure disposed at least partially within the frame and exposed on at least one side of the frame to permit bone growth into the lattice structure. The lattice structure comprises a magnesium phosphate material.

### RELATED APPLICATIONS

This application claims priority to U.S. Provisional Application No. 63/257,640 entitled “Spinal Implant with a Magnesium-Phosphate Three-Dimensional Porosity Structure,” filed on Oct. 20, 2021, the contents of which are hereby incorporated by reference in its entirety.

### TECHNICAL FIELD

The present disclosure relates to surgical implants, particularly spinal implants for use in spinal fusion applications.

### BACKGROUND

Unless otherwise indicated herein, the materials described in this section are not admitted to be prior art to the claims in this application.

A variety of different implants are used in the body. Implants used in the body to stabilize an area and promote bone ingrowth provide both stability (i.e. minimal deformation under pressure over time) and space for bone ingrowth.

Spinal fusion, also known as spondylodesis or spondylosyndesis, is a surgical technique by which two or more vertebrae are joined together. This technique is used to treat various conditions such as, for example, spinal deformities, damaged spinal discs, and vertebral fractures. Fusion may be effected by the introduction of new bone tissue between the vertebrae to be joined and the stimulation of the natural bone growth capabilities of the vertebrae themselves. In some procedures, spinal discs and/or vertebrae may be replaced with a spacer, or cage, that maintains a proper distance between vertebrae and provides a structure through which the vertebrae may grow and eventually, fuse together.

### SUMMARY

In a first aspect, the present disclosure provides a spinal implant for insertion between two adjacent vertebrae. The spinal implant includes a frame sized to be inserted between the two adjacent vertebrae. The spinal implant also includes a lattice structure disposed at least partially within the frame and exposed on at least one side of the frame to permit bone growth into the lattice structure. The lattice structure comprises a magnesium phosphate material.

In a second aspect, the present invention provides a method for securing a spinal implant between two adjacent vertebrae, the method comprising: (i) providing the spinal implant of the first aspect, (ii) forming a space between two adjacent vertebrae, and (iii) inserting the spinal implant between the two adjacent vertebrae.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a perspective view of a spinal implant, according to an exemplary embodiment.

FIG. 2 illustrates a front view of the spinal implant of FIG. 1, according to an exemplary embodiment.

FIG. 3 illustrates a side view of the spinal implant of FIG. 1, according to an exemplary embodiment.

FIG. 4 illustrates a perspective view of a spinal implant, according to an exemplary embodiment.

FIG. 5 illustrates a top view of the spinal implant of FIG. 4, according to an exemplary embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The exemplary embodiments described herein are not meant to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, “distal” with respect to a portion of the apparatus means the end of the device (when in use) nearer the treatment zone (e.g., the cavity in a structure) of the subject and the term “proximal” means the portion of the device (when in use) further away from the treatment zone of the subject and nearer the access site and the operator.

As used herein, with respect to measurements, “about” means+/−5%.

As used herein, “osteostimulative” refers to the ability of a material to improve healing of bone injuries or defects.

As used herein, “osteoconductive” refers to the ability of a material to serve as a scaffold for viable bone growth and healing.

As used herein, “osteoinductive” refers to the capacity of a material to stimulate or induce bone growth.

As used herein, “biocompatible” refers to a material that elicits no significant undesirable response when inserted into a recipient (e.g., a mammalian, including human, recipient).

As used herein, “resorbable” refers to a material's ability to be absorbed in-vivo through bodily processes. The absorbed material may turn into bone in the patient's body.

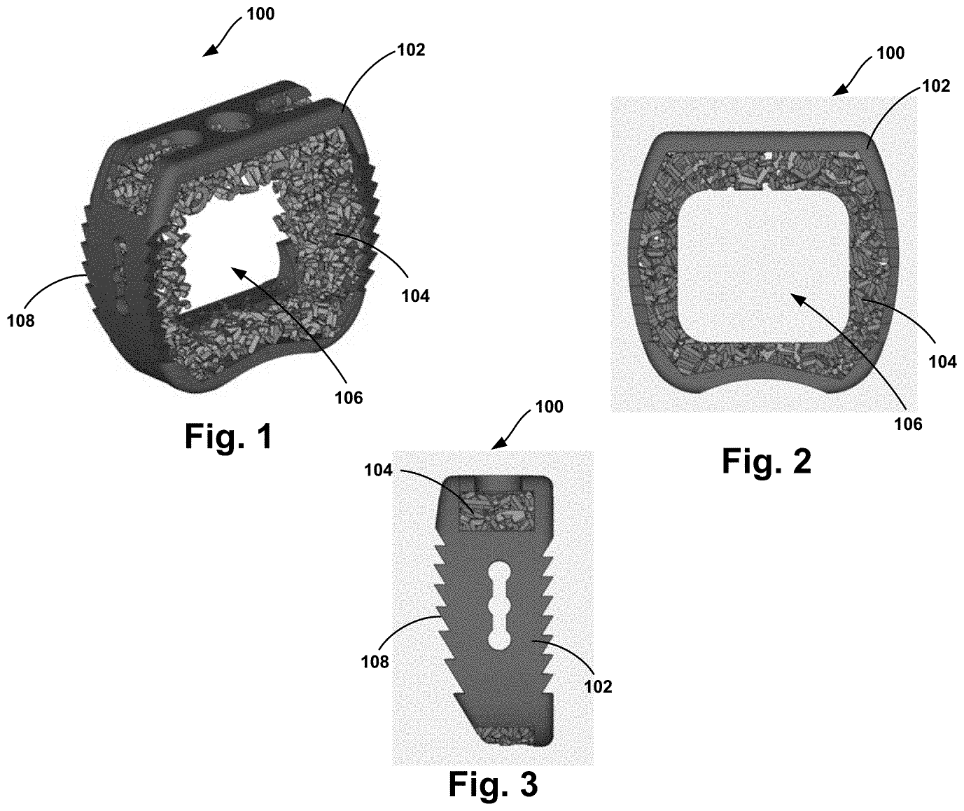

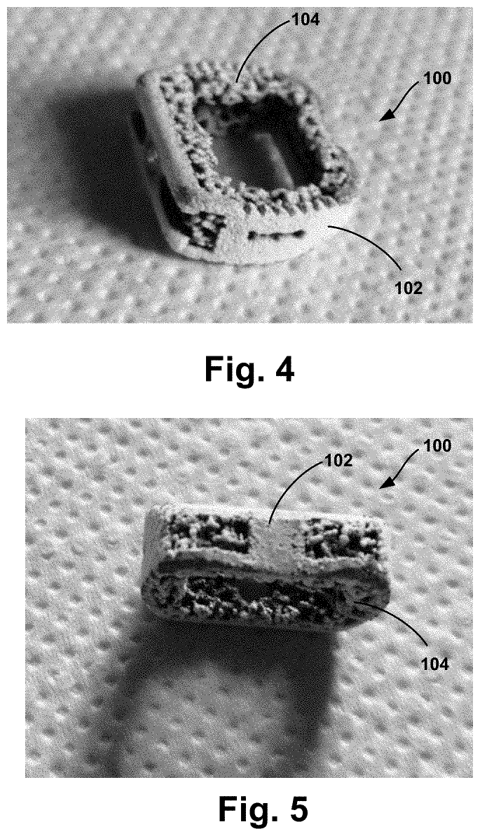

With reference to the figures, the present disclosure provides a spinal implant 100. FIG. 1 illustrates a perspective view of the spinal implant 100, including a frame 102 sized to be inserted between the two adjacent vertebrae, and a lattice structure 104 disposed at least partially within the frame 102 and exposed on at least one side of the frame 102 to permit bone growth into the lattice structure 104. The lattice structure 104 comprises a magnesium phosphate material. As used herein, “magnesium phosphate material” is a general term for salts of magnesium and phosphate appearing in several forms and several hydrates including, but not limited to, monomagnesium phosphate ((Mg(H₂PO₄)₂).xH₂O), dimagnesium phosphate ((MgHPO₄).xH₂O), and trimagnesium phosphate ((Mg₃(PO₄)₂).xH₂O).

In one example, the lattice structure 104 further includes KH₂PO₄ in an amount between about 20-70 dry weight percent, MgO in an amount between 10-50 dry weight percent, a calcium containing compound, and a poly-lactic acid. In such an example, the poly-lactic acid comprises one of Poly(L-lactic acid) PLA, poly(L, DL-lactide) PLDLA, and poly(L-lactide-co-glycolide) PLGA. Further, the lattice structure 104 may include a bioactive therapeutic agent. Such bioactive therapeutic agents may include natural or synthetic therapeutic agents such as bone morphogenic proteins (BMPs), growth factors, bone marrow aspirate, stem cells, progenitor cells, antibiotics, amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin, or other osteoconductive, osteoinductive, osteogenic, bio-active, or any other fusion enhancing material or beneficial therapeutic agent.

In one example, the lattice structure 104 further comprises a sugar, and wherein the sugar comprises one of sugar alcohols, sugar acids, amino sugars, sugar polymers glycosaminoglycans, glycolipds, sugar substitutes and combinations thereof. In some examples, the lattice structure 104 does not cover the entirety of the interior surface of the frame 102 such that there are areas of bare titanium polyetheretherketone (PEEK), polyurethane, and/or bone. In another example, the entire interior surface of the frame 102 is covered with the lattice structure 104.

The lattice structure 104 may secured to the elongated member of the orthpedic implant in a variety of ways. In one example, an energy source (e.g., a laser or electron beam) is used with a magnesium phosphate powder to build structures layer by layer, selectively sintering powder together so as to build a three dimensional shape. More particularly, a thin layer of the powder is spread out as a uniform layer, and then the energy source is used to selectively melt regions of the powder, fusing the particles together. Another layer of powder is then spread on top of the first layer, and the energy source again melts regions of the powder. This process is continued until the complete three-dimensional lattice structure 104 is built. It is possible to manufacture the lattice structure 104 such that it can be positioned within the frame 102 after manufacturing, or the lattice structure 104 may be manufactured directly onto the frame 102 of the spinal implant 100.

In another example, the lattice structure 104 is formed of sintered layers of powder that create a three-dimensional porous coating. In particular, multiple thin sheets of material may be laminated one on top of another. A pattern can be chemically etched, punched, or cut out of each of the sheets and, by altering the geometry of the pattern on each sheet, it is possible to create a porous dodecahedron or other multi-facet structure which can function as a lattice structure 104 for the spinal implant 100. More particularly, each sheet can be layered one on top of another and sintered together so as to create a porous structure. By changing the geometry of the cut-out on each layer, it is possible to create many different porous structures.

In another example, a structure similar to trabecular bone (e.g., a polyurethane foam) is coated with another material (e.g., a magnesium phosphate material) by vapor deposition, low temperature arc vapor deposition (LTAVD), chemical vapor deposition, ion beam assisted deposition and/or sputtering. The underlying structure (e.g., the polyurethane foam) may then undergo pyrolysis so as to remove the underlying structure (e.g., the polyurethane foam), leaving a magnesium phosphate based metallic lattice structure 104 which can be attached to the frame 102 (e.g., by sintering, brazing, diffusion bonding, gluing or cementing, etc.).

In one example, as shown in FIG. 1, the frame further comprises a through bore 106, and the lattice structure 104 is further exposed to the interior surface of the through bore 106. In one example, the lattice structure 104 is a non-random lattice structure. In another example, the lattice structure 104 is a random lattice structure.

The frame 102 may take a variety of forms. In one example, the frame 102 of the spinal implant 100 may comprise titanium, polyetheretherketone (PEEK), polyurethane, bone, or combinations thereof. Further, in one example, the frame 102 may include a plurality of ridges 108 that are used to secure the spinal implant 100 between two adjacent vertebrae during installation of the spinal implant 100.

In another example, the entirety of the frame 102 of the spinal implant 100 is made from a cured osteostimulative material including a polymer including poly-lactic acid and either magnesium phosphate or calcium phosphate. As used herein, “poly-lactic acid” or polylactide (PLA) is a biodegradable and bioactive thermoplastic aliphatic polyester derived from renewable resources, and may take a variety of forms including, but not limited to, poly-L-lactide (PLLA), poly-D-lactide (PDLA), and poly(L-lactide-co-D,L-lactide) (PLDLLA). As used herein, “magnesium phosphate” is a general term for salts of magnesium and phosphate appearing in several forms and several hydrates including, but not limited to, monomagnesium phosphate ((Mg(H₂PO₄)₂).xH₂O), dimagnesium phosphate ((MgHPO₄).xH₂O), and trimagnesium phosphate ((Mg₃(PO₄)₂).xH₂O). As used herein, “calcium phosphate” is a family of materials and minerals containing calcium ions (Ca²⁺) together with inorganic phosphate anions and appearing in a variety of forms including, but not limited to, monocalcium phosphate, dicalcium phosphate, tricalcium phosphate, octacalcium phosphate, amorphous calcium phosphate, dicalcium diphosphate, calcium triphosphate, hydroxyapatite, apatite, and tetracalcium phosphate.

Making the entirety of the frame 102 of the spinal implant 100 from a polymer including poly-lactic acid and either magnesium phosphate or potassium phosphate has a number of advantages. In particular, such a material allows the spinal implant 100 to be absorbed in-vivo, producing increased fixation strength and faster absorption into the body. As such, there is no void that is left behind after the spinal implant 100 is absorbed in-vivo. Instead, the spinal implant 100 is replaced with bone structure grown naturally in the body and the resulting fixation strength is very strong. As such, the frame 102 of the spinal implant 100 may be completely resorbable.

In operation, the present invention provides a method for securing an orthopedic implant to a bone, the method comprising: (a) providing the spinal implant 100 of any of the embodiments described above, (b) forming a space between two adjacent vertebrae, and (c) inserting the spinal implant 100 between the two adjacent vertebrae.

In some examples, one or more components of the spinal implant 100 described above is made via an additive manufacturing process using an additive-manufacturing machine, such as stereolithography, multi-jet modeling, inkjet printing, selective laser sintering/melting, and fused filament fabrication, among other possibilities. Additive manufacturing enables one or more components of the orthopedic implant and other physical objects to be created as intraconnected single-piece structure through the use of a layer-upon-layer generation process. Additive manufacturing involves depositing a physical object in one or more selected materials based on a design of the object. For example, additive manufacturing can generate one or more components of the orthopedic implant using a Computer Aided Design (CAD) of the orthopedic implant as instructions. As a result, changes to the design of the spinal implant 100 can be immediately carried out in subsequent physical creations of the spinal implant 100. This enables the components of the spinal implant 100 to be easily adjusted or scaled to fit different types of applications (e.g., for use in various vertebrae sizes). In one particular example, the step of securing the lattice structure 104 to the frame 102 of the spinal implant 100 comprises performing an additive-manufacturing process to deposit the lattice structure 104 on the interior surface of the frame 102.

The layer-upon-layer process utilized in additive manufacturing can deposit one or more components of the spinal implant 100 with complex designs that might not be possible for devices assembled with traditional manufacturing. In turn, the design of the spinal implant 100 can include aspects that aim to improve overall operation. For example, the design can incorporate physical elements that help redirect stresses in a desired manner that traditionally manufactured devices might not be able to replicate.

Additive manufacturing also enables depositing one or more components of the spinal implant 100 in a variety of materials using a multi-material additive-manufacturing process. In such an example, the frame 102 may be made from a first material and the lattice structure 104 may be made from a second material that is different than the first material. In another example, both the frame 102 and the lattice structure 104 are made from the same material. Other example material combinations are possible as well. Further, one or more components of the spinal implant 100 can have some layers that are created using a first type of material and other layers that are created using a second type of material. In addition, various processes are used in other examples to produce one or more components of the spinal implant 100. These processes are included in table 1.

TABLE 1

| | |
|---|---|
| DEP | Direct Energy Deposition |
| DMLS | Direct Metal Laser Sintering |
| DMP | Direct Metal Printing |
| EBAM | Electron Beam Additive Manufacturing |
| EBM | Electron Beam Leting |
| EBPD | Electron Beam Powder Bed |
| FDM | Fused Deposition Modeling |
| IPD | Indirect Power Bed |
| LCT | Laser Cladding Technology |
| LDT | Laser Deposition Technology |
| LDW | Laser Deposition Welding |
| LDWM | Laser Deposition Welding with integrated Milling |
| LENS | Laser Engineering Net Shape |
| LFMT | Laser Freeform Manufacturing Technology |
| LMD-p | Laser Metal Deposition-powder |
| LMD-w | Laser Metal Deposition-wire |
| LPB | Laser Powder Bed |
| LPD | Laser Puddle Deposition |
| LRT | Laser Repair Technology |
| PDED | Powder Directed Energy Deposition |
| SLA | Stereolithography |
| SLM | Selective Laser Melting |
| SLS | Selective Laser Sintering |
| SPD | Small Puddle Deposition |

Each of the components of the spinal implant 100 described above may represent a module, a segment, or a portion of program code, which includes one or more instructions executable by a processor or computing device for creating such devices using an additive-manufacturing system. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Because many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A spinal implant for insertion between two adjacent vertebrae, the spinal implant comprising:
a frame sized to be inserted between the two adjacent vertebrae; and
a lattice structure disposed at least partially within the frame and exposed on at least one side of the frame to permit bone growth into the lattice structure, wherein the lattice structure comprises a magnesium phosphate material.

2. The spinal implant of claim 1, wherein the lattice structure further includes KH₂PO₄ in an amount between about 20-70 dry weight percent, MgO in an amount between 10-50 dry weight percent, a calcium containing compound, and a poly-lactic acid.

3. The spinal implant of claim 2, wherein the poly-lactic acid comprises one of Poly(L-lactic acid) PLA, poly(L, DL-lactide) PLDLA, and poly(L-lactide-co-glycolide) PLGA.

4. The spinal implant of claim 2, wherein the lattice structure further includes a bioactive therapeutic agent.

5. The spinal implant of claim 4, wherein the bioactive therapeutic agent comprises one of amikacin, butirosin, dideoxykanamycin, fortimycin, gentamycin, kanamycin, lividomycin, neomycin, netilmicin, ribostamycin, sagamycin, seldomycin and epimers thereof, sisomycin, sorbistin, spectinomycin and tobramycin.

6. The spinal implant of claim 2, wherein the lattice structure further comprises a sugar, and wherein the sugar comprises one of sugar alcohols, sugar acids, amino sugars, sugar polymers glycosaminoglycans, glycolipds, sugar substitutes and combinations thereof.

7. The spinal implant of claim 1, wherein the frame further comprises a through bore, and wherein the lattice structure is further exposed to the through bore.

8. The spinal implant of claim 1, wherein the frame comprises titanium, polyetheretherketone (PEEK), polyurethane, bone, or a cured osteostimulative material.

9. The spinal implant of claim 1, wherein the frame is resorbable.

10. The spinal implant of claim 1, wherein the lattice structure is a non-random lattice structure.

11. The spinal implant of claim 1, wherein the lattice structure is a random lattice structure.

12. The spinal implant of claim 1, wherein the lattice structure is formed of sintered layers of powder that create a three-dimensional porous coating.

13. The spinal implant of claim 1, wherein the lattice structure is formed via a chemical vapor deposition process.

14. A method for securing a spinal implant between two adjacent vertebrae, the method comprising:
providing the spinal implant of claim 1;
forming a space between two adjacent vertebrae; and
inserting the spinal implant between the two adjacent vertebrae.
